(19) United States
(12) Patent Application Publication
(10) Pub. No.: US 2017/0208771 A1
(43) Pub. Date: Jul. 27, 2017

(54) Method for Separating Swine Waste

(71) Applicant: Timothy H Craig, Sanford, NC (US)

(72) Inventor: Timothy H Craig, Sanford, NC (US)

(21) Appl. No.: 15/413,223

(22) Filed: Jan. 23, 2017

Related U.S. Application Data
(60) Provisional application No. 62/281,481, filed on Jan. 21, 2016.

(51) Int. Cl. A01K 1/01; B01D 33/72; B01D 33/80; C12P 5/02; E04H 7/02; A01K 1/015; A01G 1/00; C10L 3/08; B01D 33/048; A01K 1/02

(52) U.S. Cl. CPC A01K 1/0135; B01D 33/048; B01D 33/72; B01D 33/801; A01K 1/0103; A01K 1/02; E04H 7/02; A01K 1/0155; A01G 1/001; C10L 3/08; C12P 5/023; C10L 2290/26

(57) ABSTRACT

A method for separating swine waste using at least one perforated conveyor belt and at least one fluid trough. A quantity of swine waste is collected through a slatted floor of a barn into at least one funneling channel. The quantity of swine waste is gravity-fed through the at least one funneling channel onto at least one perforated conveyor belt. Liquid waste is strained from the quantity of swine waste on at least one perforated conveyor belt into the fluid trough, retaining solid waste on the at least one perforated conveyor belt. The solid waste is then transported into a solid-waste storage vessel with the at least one perforated conveyor belt. Simultaneously, the liquid waste is gravity fed into a liquid-waste containment vessel from the at least one fluid trough. The solid waste and the liquid waste are stored to be processed later.

The current application claims a priority to the U.S. Provisional Patent application Ser. No. 62/281,481 filed on Jan. 21, 2016. The current application is filed on Jan. 23, 2017 while Jan. 21, 2017 was on a weekend.

### FIELD OF THE INVENTION

The present invention relates generally to a method for managing swine waste. More specifically, the present invention separates solid hog excrement from liquid hog excrement to be used separately in beneficial applications.

### BACKGROUND OF THE INVENTION

Currently, most hog farms use a lagoon to store manure before the manure is applied to growing crops through an irrigation system, a drag hose injection system, or by tanker injection. These methods are very effective ways to introduce the fertilizer to growing crops in order to impart nutrients found within the manure to the growing crops. Waste excrement is flushed into the lagoon to undergo anaerobic respiration. The use of lagoons, however, have been linked with adverse environmental and health effects from gas emissions and waste overflowing from the lagoon due to weather conditions releasing harmful substances to the surroundings.

The objective of the present invention is to provide a process to improve separation and containment of swine waste excrement. The waste excrement is separated into solid waste and liquid waste for specific applications. The present invention works by using at least one perforated conveyor belt to transport solid waste from a structure, whilethe liquid waste is caught in a gutter system and flows into containment.

### BRIEF DESCRIPTION OF THE DRAWINGS

FIG. 1 is a flow chart showing the general process of the present invention.

FIG. 2 is a flow chart showing the production of methane and electricity from the combustion of the methane.

FIG. 3 is flow chart showing the conversion of solid waste into composted top soil and the storage of liquid waste.

FIG. 4 is a flow chart detailing a timer controller to control a plurality of rollers and a belt of the at least one perforated conveyor belt.

FIG. 5 is a schematic view for a cross section for a barn across a width of the at least one perforated conveyor belt to detail the present invention.

FIG. 6 is a schematic view for a cross section for a barn along a length of the at least one perforated conveyor belt to detail the present invention.

### DETAIL DESCRIPTIONS OF THE INVENTION

All illustrations of the drawings are for the purpose of describing selected versions of the present invention and are not intended to limit the scope of the present invention.

The present invention is a method for separating swine waste into solid waste and liquid waste for subsequent processing. The present invention makes the handling of solid waste and liquid waste easier and more efficient than current lagoon systems. Lagoon systems store the swine waste in open containment vessels that are prone to overflowing either by excess flush water or heavy precipitation. An overflowing lagoon has the potential to become hazardous to the health of people local to the containment vessel and the local environment. Traditionally, from a lagoon system, the manure is applied to growing crops through irrigation systems, drag hose injection systems, or tanker injection systems; however, due to the use of water to suspend the manure in a slurry to be pumped to growing crops, and precipitation, the concentration of nutrients is diluted.

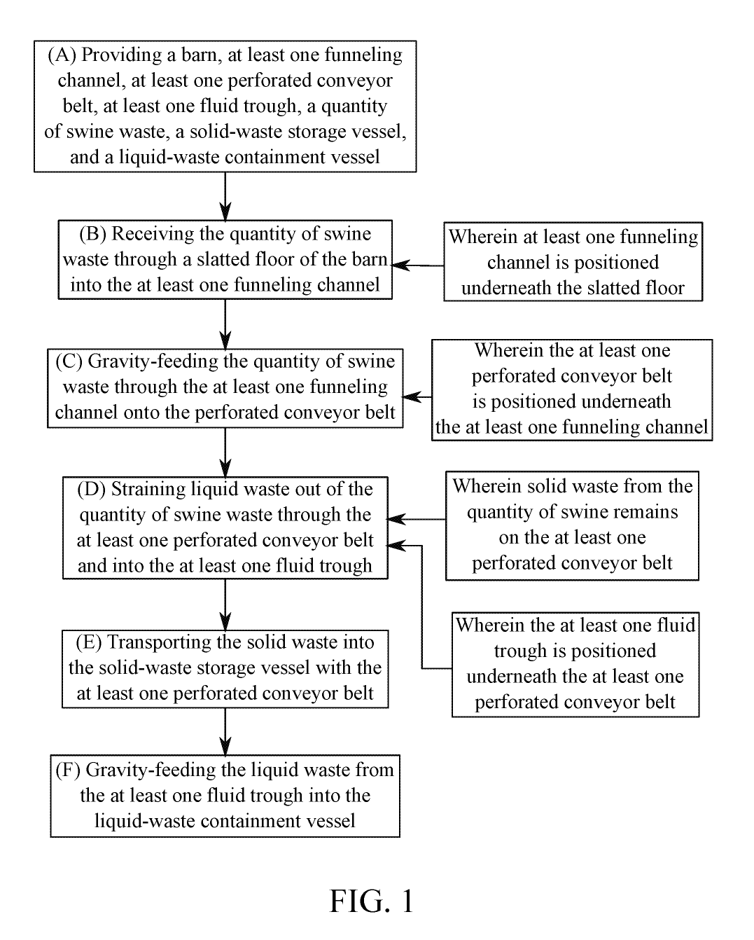

As previously mentioned, the present invention is a method for separating swine waste within new barns or remodeled barns. In order to execute the present invention, a barn 1, at least one funneling channel 2, at least one perforated conveyor belt 3, at least one fluid trough 4, a quantity of swine waste 5, a solid-waste storage vessel 6, and a liquid-waste containment vessel 7 are required, in accordance to FIG. 1. The barn 1 is a structure to house and protect livestock from the elements. The at least one funneling channel 2 receives and converges the quantity of swine waste 5 from the barn 1 onto the at least one perforated conveyor belt 3. The at least one perforated conveyor belt 3 transports solid waste 10 from the quantity of swine waste 5 from the barn 1 into the solid-waste storage vessel 6, as well as, strains liquid waste 9 from the quantity of swine waste 5. The solid-waste storage vessel 6 contains the solid waste 10 for later use. The at least one fluid trough 4 receives the liquid waste 9 from the at least one perforated conveyor belt 3 to transport the liquid waste 9 to the liquid-waste containment vessel 7.

The liquid-waste containment vessel 7 collects the liquid waste 9. A roof of the barn 1 prevents precipitation from diluting the liquid waste 9 by sheltering the slatted floor 8 from precipitation.

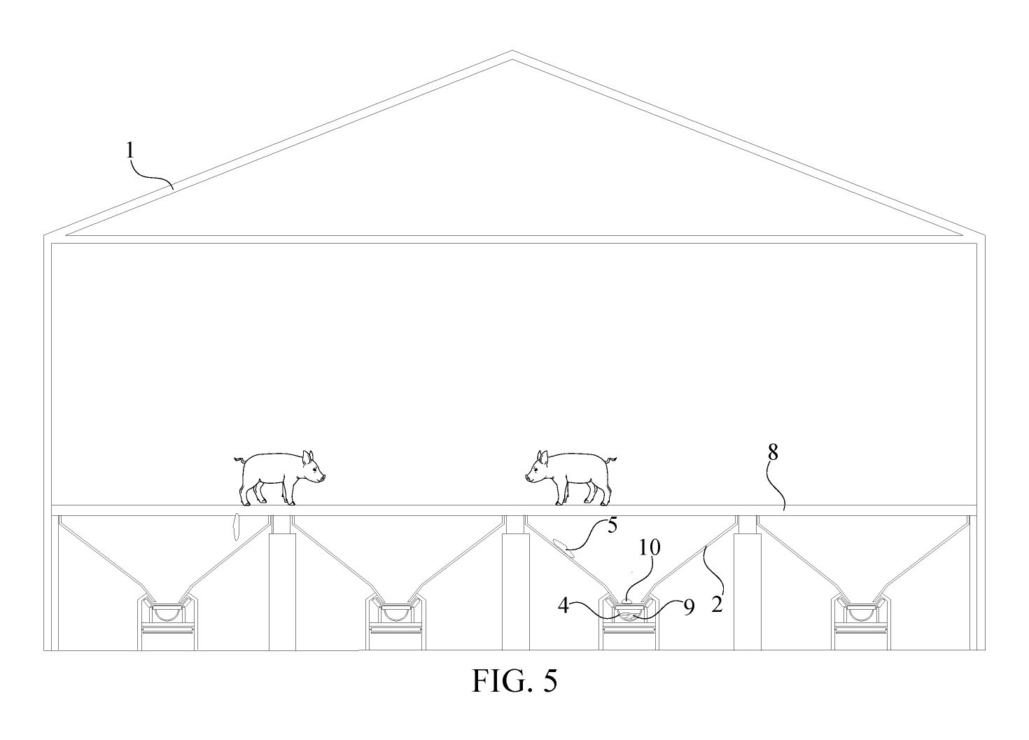

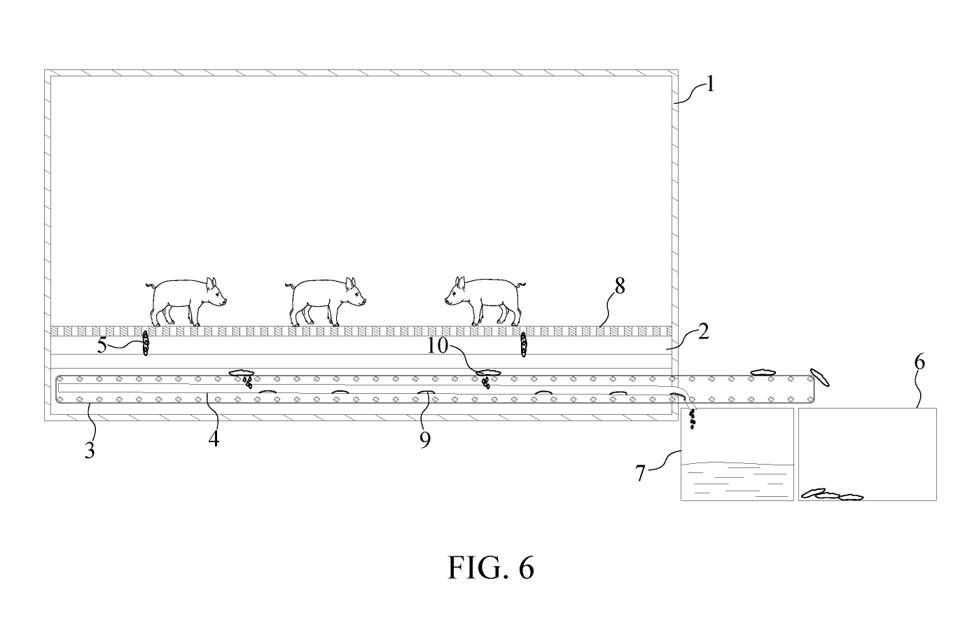

Initially, the quantity of swine waste 5 is received through a slatted floor 8 of the barn 1, in accordance to FIG. 1, FIG. 5 and FIG. 6. The quantity of swine waste 5 is a product of the digestion of the livestock housed within the barn 1. The slatted floor 8 allows the quantity of swine waste 5 to pass through the floor of the barn 1 as the livestock excretes the quantity of swine waste 5, reducing the accumulation of swine waste within the living space of the livestock. The quantity of swine waste 5 is then gravity-fed through the at least one funneling channel 2, that is positioned underneath the slatted floor 8, onto the at least one perforated conveyor belt 3, that is positioned underneath the at least one funneling channel 2. Liquid waste 9 is then strained out of the quantity of swine waste 5 through the at least one perforated conveyor belt 3 and into the at least one fluid trough 4, positioned underneath the at least one perforated conveyor belt 3. The solid waste 10 from the quantity of swine waste 5 remains on the at least one perforated conveyor belt 3, therefore, separating the solid waste 10 from the liquid waste 9. The solid waste 10 is transported into the solid-waste storage vessel 6 with the at least one perforated conveyor belt 3 to store the solid waste 10 for further processing into manure. The liquid waste 9 is simultaneously gravity-fed into the liquid-waste containment vessel 7 to collect the liquid waste 9 for further processing.

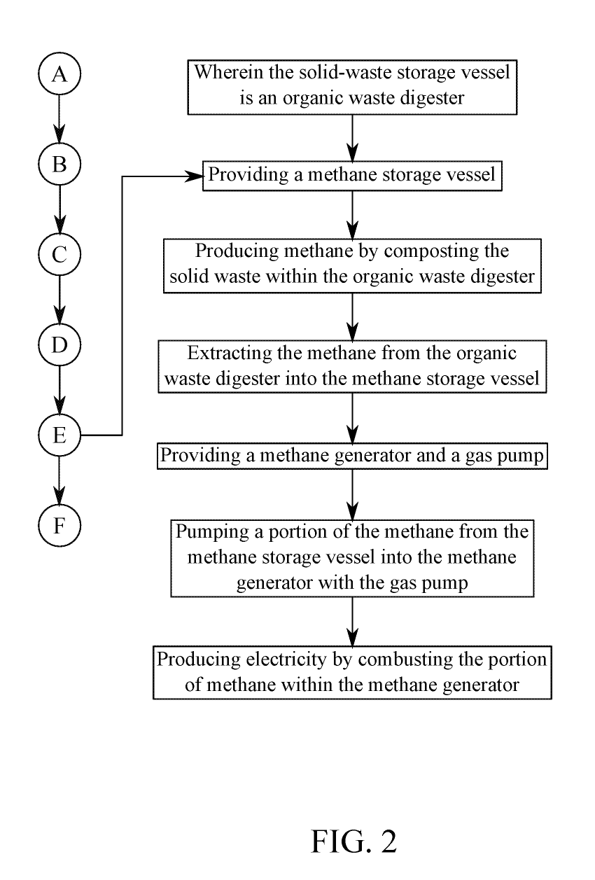

In accordance to the preferred embodiment of the present invention, the solid-waste storage vessel 6 is an organic waste digester, detailed in FIG. 2. An organic waste digester converts organic waste into useable fertilizer while producing biogas, more specifically methane, as a byproduct. The organic waste digester is selected from a group including mesophilic digesters, thermophilic digesters, and combinations thereof. The present invention requires a methane storage vessel to collect the methane for storage before being transported offsite or being processed onsite. Methane is produced by composting the solid waste 10 within the organic waste digester. The methane is then extracted from the organic waste digester into the methane storage vessel.

In some embodiments of the present invention, a methane generator and a gas pump are incorporated to produce electricity, as shown in FIG. 2. A portion of the methane is pumped from the methane storage vessel into the methane generator with the gas pump. Electricity is then produced by combusting the portion of methane within the methane generator. The electricity produced using the methane generator is then used to power or subsidize power to the at least one perforated conveyor belt 3, any lighting fixtures and receptacles within the barn 1, or other electrical applications for the present invention. Additionally, the electricity can provide power to a local electrical grid.

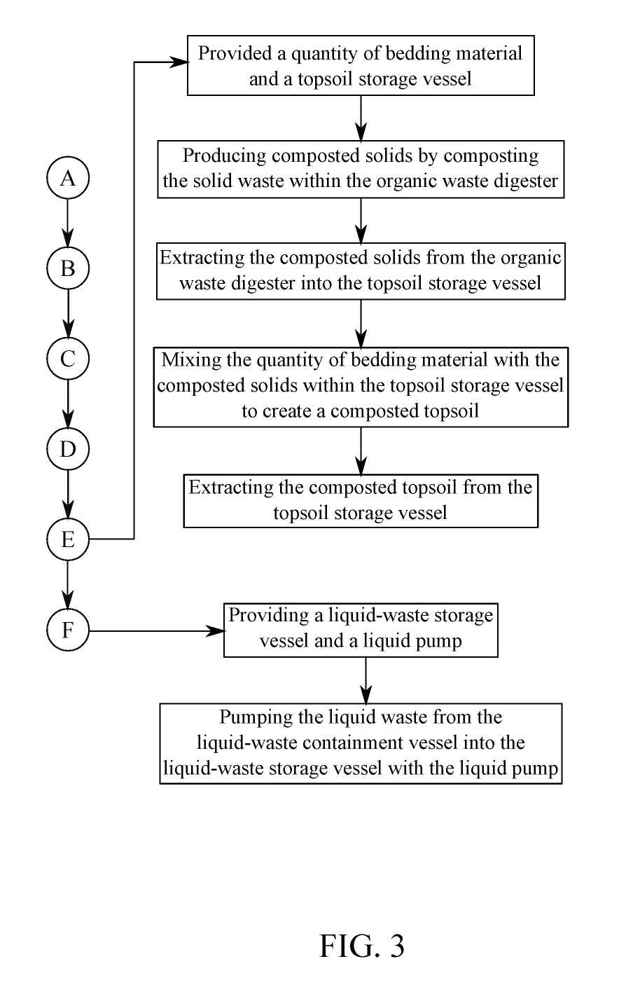

In some embodiments of the present invention, a liquid-waste storage vessel is required to store the liquid waste 9 before the liquid waste 9 is processed and a liquid pump to transport the liquid waste 9. The liquid waste 9 collected within the liquid-waste containment vessel 7 is pumped into the liquid-waste storage vessel with the liquid pump, as shown in FIG. 3. From the liquid-waste storage vessel, the liquid waste 9 is applied to irrigation fields at agronomic rates. Agronomic rates ensure the liquid waste 9 is applied based on the quantity of nutrients in the liquid waste 9 and the quantity of nutrients needed by the crop being grown.

Further, in accordance to the preferred embodiment, the present invention uses a quantity of bedding materials to produce topsoil from the solid waste 10 in a topsoil storage vessel, detailed in FIG. 3. The quantity of bedding materials includes, but is not limited to, sawdust, wood shavings, or other waste plant matter. Composted solids are produced by composting the solid waste 10 within the organic waste digester. The composted solids are then extracted from the organic waste digester into the topsoil storage vessel. The quantity of bedding materials is then mixed with the composted solids within the topsoil storage vessel to create a composted topsoil. The composted top soil is a soil that provides vital nutrients to plants when the composted top soil is distributed around the base of the plants or plant seeds. The composted topsoil is extracted from the topsoil storage vessel to fertilize a desired area.

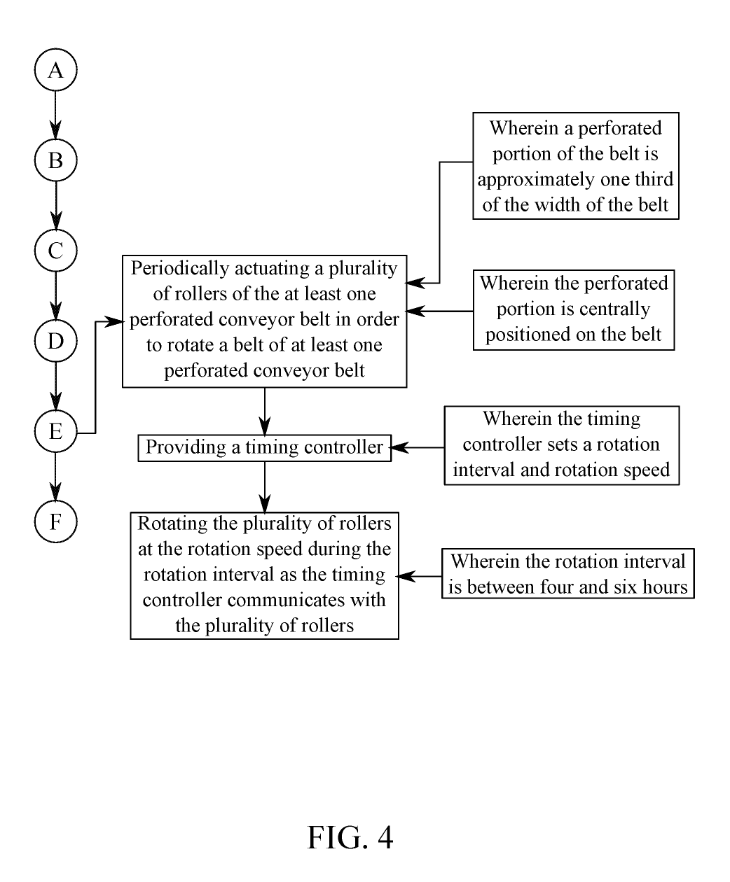

In order to transport the solid waste 10 from the at least one perforated conveyor belt 3 to the solid-waste storage vessel 6, a plurality of rollers of the at least one perforated conveyor belt 3 is actuated periodically in order to rotate a belt of the at least one perforated conveyor belt 3, in accordance to FIG. 4. The belt is trained around the plurality of rollers in order to drive the belt. A timing controller is required for the present invention to control the behavior of the at least one perforated conveyor belt 3. The timing controller sets a rotation interval and a rotation speed. The rotation interval determines the duration that the plurality of rollers is rotated and the rotation speed is the rate at which the plurality of rollers is rotated. The plurality of rollers is rotated at the rotation speed during the rotation interval as the timing controller communicates with the plurality of rollers. The rotation interval is preferred to be every four to six hours in order to transport the solid waste 10 on the belt to the solid-waste storage vessel 6 and to prevent accumulation of the solid waste 10 on the belt.

In accordance to FIG. 4, a perforated portion of the belt is approximately one third of the width of the belt in order to provide sufficient surface area to allow the liquid waste 9 to easily pass through the at least one perforated conveyor belt 3. The perforated portion is preferred to be centrally positioned on the belt as the liquid waste 9 is focused towards the center of the at least one perforated conveyor belt 3 from the at least one funneling channel 2 in order to maximize the collection of solid waste 10 as the quantity of swine waste 5 impacts the belt from the at least one funneling channel 2.

Although the invention has been explained in relation to its preferred embodiment, it is to be understood that many other possible modifications and variations can be made without departing from the spirit and scope of the invention as hereinafter claimed.

What is claimed is:

1. A method for separating swine waste comprises the steps of:
providing a barn, at least one funneling channel, at least one perforated conveyor belt, at least one fluid trough, a quantity of swine waste, a solid-waste storage vessel, and a liquid-waste containment vessel;
receiving the quantity of swine waste through a slatted floor of the barn into the at least one funneling channel;
gravity-feeding the quantity of swine waste through the at least one funneling channel onto the perforated conveyor belt;
straining liquid waste out of the quantity of swine waste through the at least one perforated conveyor belt and into the at least one fluid trough, wherein solid waste from the quantity of animal remains on the at least one perforated conveyor belt;
transporting the solid waste into the solid-waste storage vessel with the at least one perforated conveyor belt; and
gravity-feeding the liquid waste from the at least one fluid trough into the liquid-waste containment vessel.

2. The method for separating swine waste, as claimed in claim 1, comprises the steps of:
wherein the solid-waste storage vessel is an organic waste digester;
providing a methane storage vessel;
producing methane by composting the solid waste within the organic waste digester; and
extracting the methane from the organic waste digester into the methane storage vessel.

3. The method for separating swine waste, as claimed in claim 2, comprises the steps of:
providing a methane generator and a gas pump;
pumping a portion of the methane from the methane storage vessel into the methane generator with the gas pump; and
producing electricity by combusting the portion of methane within the methane generator.

4. The method for separating swine waste, as claimed in claim 1, comprises the steps of:
providing a liquid-waste storage vessel and a liquid pump; and
pumping the liquid waste from the liquid-waste containment vessel into the liquid-waste storage vessel with the liquid pump.

5. The method for separating swine waste, as claimed in claim 1, comprises the steps of:
provided a quantity of bedding material and a topsoil storage vessel;
producing composted solids by composting the solid waste within the organic waste digester;
extracting the composted solids from the organic waste digester into the topsoil storage vessel;
mixing the quantity of bedding material with the composted solids within the topsoil storage vessel to create a composted topsoil; and
extracting the composted topsoil from the topsoil storage vessel.

6. The method for separating swine waste, as claimed in claim 1, comprises the step of:
periodically actuating a plurality of rollers of the at least one perforated conveyor belt in order to rotate a belt of at least one perforated conveyor belt.

7. The method for separating swine waste, as claimed in claim 6, comprises the steps of:
providing a timing controller, wherein the timing controller sets a rotation interval and rotation speed; and
rotating the plurality of rollers at the rotation speed during the rotation interval as the timing controller communicates with the plurality of rollers.

8. The method for separating swine waste, as claimed in claim 7, wherein the rotation interval is between four and six hours.

9. The method for separating swine waste, as claimed in claim 6, wherein a perforated portion of the belt is approximately one third of the width of the belt, and wherein the perforated portion is centrally positioned on the belt.

10. The method for separating swine waste, as claimed in claim 1, wherein at least one funneling channel is positioned underneath the slatted floor.

11. The method for separating swine waste, as claimed in claim 1, wherein the at least one perforated conveyor belt is positioned underneath the at least one funneling channel.

12. The method for separating swine waste, as claimed in claim 1, wherein the at least one fluid trough is positioned underneath the at least one perforated conveyor belt.
